(19) United States
(12) Patent Application Publication
(10) Pub. No.: US 2024/0115535 A1
(43) Pub. Date: Apr. 11, 2024

(54) Methods for Inhibiting the Progression of Neurodegenerative Diseases

(71) Applicants: Peter MILNER, Los Altos, CA (US); Mikhail Sergeevich SHCHEPINOV, Los Altos, CA (US); Retrotope, Inc., Suite 201, CA (US)

(72) Inventors: Peter Milner, Los Altos, CA (US); Mikhail Sergeevich Shchepinov, Oxford (GB)

(21) Appl. No.: 18/275,972

(22) PCT Filed: Feb. 7, 2022
(86) PCT No.: PCT/US2022/015535

§ 371 Date: Aug. 4, 2023

Related U.S. Application Data
(63) Continuation-in-part of application No. 17/391,909, filed on Aug. 2, 2021, now Pat. No. 11,491,130.
Application No. 17/391,909 is a continuation-in-part of application No. 17/169,271, filed on Feb. 5, 2021, now Pat. No. 11,351,143.

(51) Int. Cl. A61K 31/201; A61P 25/28; G01N 33/50; G01N 33/555

(52) U.S. Cl. CPC A61K 31/201; A61P 25/28; G01N 33/5044; G01N 33/555

(57) ABSTRACT

Disclosed are methods for inhibiting the progression of neurodegenerative disease. The methods include administering to a patient suffering from such a disease a composition comprising deuterated arachidonic acid or an ester thereof.

### CROSS-REFERENCE TO RELATED APPLICATIONS

This application is a continuation-in-part of and claims the benefit U.S. patent application Ser. No. 17/169,271 filed Feb. 5, 2021 and U.S. patent application Ser. No. 17/391,909, filed Aug. 2, 2021, both of which are incorporated herein by reference in their entirety.

### TECHNICAL FIELD

Disclosed are methods for inhibiting the progression of neurodegenerative diseases in humans. The methods use a specific dosing regimen to treat a patient suffering from a neurodegenerative disease treatable with a deuterated arachidonic acid or a prodrug thereof. In particular, the dosing regimen provides for rapid onset to a therapeutic concentration in vivo of deuterated arachidonic acid at a level where the progression of the disease is markedly reduced.

### BACKGROUND

There are a number of debilitating neurodegenerative diseases in humans which despite the best efforts of researchers remain incurable and often fatal. As such, the attending clinician attempts to slow the progression of the disease and, where possible, maintain the quality of life for the patient for as long as possible. Examples of such neurodegenerative diseases include the following:
1. amyotrophic lateral sclerosis (ALS) which is a late-onset, progressive neurological disease with its corresponding pathological hallmarks including progressive muscle weakness, muscle atrophy and spasticity all of which reflect the degeneration and death of upper or lower motor neurons. Once diagnosed, most patients undergo a rapid rate of disease progression terminating in death typically within 3 to 4 years with some patients succumbing even earlier;
2. tauopathy is a subgroup of Lewy body diseases or proteinopathies and comprises neurodegenerative conditions involving the aggregation of tau protein into insoluble tangles. These aggregates/tangles form from hyperphosphorylation of tau protein in the human brain. Specific conditions related to tauopathy include, but are not limited to, argyrophilic grain disease (AGD), chronic traumatic encephalopathy (CTE), corticobasal degeneration (CBD), frontotemporal dementia and parkinsonism linked to chromosome 17 (FTDP-17), ganglioglioma, gangliocytoma, lipofuscinosis, lytico-bodig disease, meningioangiomatosis, pantothenate kinase-associated neurodegeneration (PKAN), Pick's disease, postencephalitic parkinsonism, primary age-related tauopathy (PART), Steele-Richardson-Olszewski syndrome (SROS), and subacute sclerosing panencephalitis (SSPE). Wang et al., Nature Rev. Neurosci. 2016; 17:5 and Arendt et al., Brain Res. Bulletin 2016; 126:238. Tauopathies often overlap with synucleinopathies.
3. Steele-Richardson-Olszewski syndrome or progressive supranuclear palsy (PSP) is one example of a neurodegenerative disease mediated at least in part by tauopathy and involves the gradual deterioration and death of specific volumes of the brain. The condition leads to symptoms including loss of balance, slowing of movement, difficulty moving the eyes, and dementia. A variant in the gene for tau protein called the H1 haplotype, located on chromosome 17, has been linked to PSP. Besides tauopathy, mitochondrial dysfunction seems to be a factor involved in PSP. Especially, mitochondrial complex I inhibitors are implicated in PSP-like brain injuries;
4. Huntington's disease is a fatal genetic disorder that causes the progressive breakdown of nerve cells in the brain;
5. Corticobasal disorder (CBD) is a rare neurodegenerative disease characterized by gradual worsening problems with movement, speech, memory and swallowing. It's often also called corticobasal syndrome (CBS). CBD is caused by increasing numbers of brain cells becoming damaged or dying over time;
6. Frontotemporal dementia (FTD) is a neurodegenerative disease and a common cause of dementia. It is characterized by a group of disorders that occur when nerve cells in the frontal temporal lobes of the brain are lost thereby causing the lobes to shrink. FTD can affect behavior, personality, language, and movement;
7. Nonfluent variant primary progressive aphasia (nfvPPA) occurs as a result of a build-up of one of two proteins, either tau or TPD-43, usually in the front left part of the brain. That part of the brain controls speech and language. As more of the protein builds up in those brain cells, the cells lose their ability to function and eventually die. As more cells die, the affected portion of the brain shrinks; and
8. Late onset Tay-Sachs is a very rare genetic neurodegenerative disease in which fatty compounds, called gangliosides, do not break down fully because the body produces too little of the enzyme hexosaminidase A (or hex A). Over time, gangliosides build up in the brain and damage brain nerve cells. This affects a person's mental functioning.

There remains a need for treatments for these and other neurodegenerative diseases.

### SUMMARY

In one embodiment, methods are disclosed that are designed to significantly attenuate the progression of neurodegenerative diseases treatable by administration of deuterated arachidonic acid. In one embodiment, methods are disclosed that significantly attenuate the progression of neurodegenerative diseases treatable by administration of deuterated arachidonic acid (D-arachidonic acid), e.g. 7,7,10,10,13,13-D6-arachidonic acid, or an ester thereof as defined below.

Such administration is delivered with a dosing regimen that comprises both a loading regimen and a maintenance regimen. The loading regimen ensures that there is a rapid onset to therapeutic levels of the deuterated arachidonic acid in vivo to attenuate disease progression thereby retaining more functionality in the patient as compared to dosing regimens that require longer periods of time to achieve therapeutic levels. The maintenance dose ensures that the therapeutic levels of deuterated arachidonic acid are maintained in the patient during therapy.

In one embodiment, deuterated arachidonic acid or an ester thereof is administered such that upon ingestion and absorption, in vivo deesterification of the ester. This is followed by vasculature transport and systemic uptake with preferential delivery to the brain.

Without being limited by theory, once generated or administered, the deuterated arachidonic acid is systemically absorbed into cells such as the cell membrane and the mitochondria. In neurons, this deuterated arachidonic acid stabilizes these cells against oxidative damage. This, in turn, stops the cascade of lipid peroxidation, thereby minimizing damage to the motor neurons. When concentrations of this deuterated arachidonic acid reach a therapeutic level in the motor neurons, the disease progression of neurodegenerative diseases is significantly attenuated.

The methods described herein provide for rapid onset of a therapeutic concentration of deuterated arachidonic acid in vivo in order to minimize unnecessary loss of functionality in the treated patients suffering from a neurodegenerative disease. In one embodiment, there is provided a method for reducing disease progression of a neurodegenerative disease in an adult patient treatable with deuterated arachidonic acid while providing for rapid onset of therapy, the method comprising administering a dosing regimen that comprises a primer or loading dose and a maintenance dose. In an embodiment, the primer or loading dose comprises periodic administration of deuterated arachidonic acid or an ester thereof. In an embodiment, the primer or loading dose is continued for at least about 30 days and, in some cases, to at least about 45 days. The primer or loading dose is designed to rapidly achieve a therapeutic concentration of deuterated arachidonic acid in vivo thereby reducing the rate of disease progression. In an embodiment, after completion of the primer or loading dose, a maintenance dose is periodically administered. In an embodiment, the maintenance dose is no more than about 65% of the primer or loading dose. In an embodiment, the therapeutic concentration of deuterated arachidonic acid is maintained in vivo such that a reduced rate of disease progression is maintained.

In one embodiment, there is provided a method for reducing disease progression of a neurodegenerative disease in an adult patient treatable with D-arachidonic acid while providing for rapid onset of therapy, the method comprising administering 7,7,10,10,13,13-D6-arachidonic acid or an ester thereof to the patient with a dosing regimen that comprises a primer or loading dose and a maintenance dose. In an embodiment, the primer or loading dose comprises periodic administration of 7,7,10,10,13,13-D6-arachidonic acid or an ester thereof. In an embodiment, the primer or loading dose comprises about 0.05 to 5 about grams or about 0.5 grams to about 5 grams of 7,7,10,10,13,13-D6-arachidonic acid or an ester thereof per day. In an embodiment, the primer or loading dose is continued for at least about 24 days to about 45 days, e.g., to rapidly achieve a therapeutic concentration of 7,7,10,10,13,13-D6-arachidonic acid in vivo, thereby reducing the rate of disease progression. In an embodiment, after completion of the primer or loading dose, the maintenance dose is periodically administered. In an embodiment no more than about 70% of the primer or loading dose of 7,7,10,10,13,13-D6-arachidonic acid or an ester thereof per day is administered. In an embodiment, the therapeutic concentration of 7,7,10,10,13,13-D6-arachidonic acid or an ester thereof is maintained in vivo such that a reduced rate of disease progression is maintained.

In an embodiment, the reduced rate of disease progression is evaluated when compared to the rate of disease progression measured prior to initiation of said method. In an embodiment, each of said neurodegenerative diseases is mediated at least in part by lipid peroxidation of polyunsaturated fatty acids in neurons of the patient suffering from said neurodegenerative disease.

In one embodiment, said neurodegenerative disease is amyotrophic lateral sclerosis, Huntington's Disease, progressive supernuclear palsy (PSP), APO-e4 Alzheimer's Disease, corticobasal disorder (CBD), frontotemporal dementia (FTD), nonfluent variant primary progressive aphasia (nfvPPA), other tauopathies, or late onset Tay-Sachs.

In one embodiment, said periodic administration of the primer or loading dose comprises administration of deuterated arachidonic acid for at least 5 days per week and preferably 7 days a week.

In one embodiment, the periodic administration of the maintenance dose of arachidonic acid or an ester thereof per day comprises no more than 55% of the primer or loading dose which is administered at least once a month. In another embodiment, the maintenance dose comprises no more than 35% of the primer or loading dose which is administered at least once a month.

In one embodiment, the periodic administration of the maintenance dose is calibrated to be an amount of 7,7,10,10,13,13-D6-arachidonic acid or an ester thereof sufficient to replace the amount of 7,7,10,10,13,13-D6-arachidonic acid removed from the body.

In one embodiment, the percent reduction in the rate of disease progression is determined by:
measuring a natural rate of disease progression in a patient or an average natural rate of disease progression in a cohort of patients prior to initiation of therapy per the methods described herein;
measuring the rate of disease progression in said patient or cohort of patients during a period of compliance with the periodic administration of both the loading step and the maintenance step; and
after a set period of time from the start of therapy, calculating the difference between the natural rate and the rate during the period of compliance, dividing the difference by the rate of disease progression during the natural history of the patient, and multiplying by 100.

In one embodiment, the set period of time is between about 1 month and about 24 months, for example about 3 months, about 6 months or about 12 months, or about 18 months or about 24 months.

In one embodiment, the methods described herein further comprise restricting the patient's consumption of excessive dietary polyunsaturated fatty acids during administration of said primer and said maintenance doses.

In one embodiment, there is provided a kit of parts comprising a set of capsules each comprising a partial primer or loading dose of deuterated arachidonic acid or an ester thereof such that two or more of said capsules comprise a complete loading dose per day.

In one embodiment, there is provided a kit of parts comprising a set of capsules each comprising a partial maintenance dose of deuterated arachidonic acid or an ester thereof such that two or more of said capsules comprise a complete maintenance dose per day.

In one embodiment, the percent change between the rate of disease progression occurring during the natural history of the patient and the decrease in the rate of disease progression during therapy is at least 25%, at least 30%, preferably at least 40%, more preferably at least 65% and most preferably greater than 70% or 80% after 1 or 3 months. Accordingly, in some embodiments, methods disclosed herein provide for determining a percent reduction in the rate of disease progression by (i) determining a natural rate of disease progression in a patient or an average natural rate of disease progression in a cohort of patients, (ii) determining the rate of disease progression in the patient or cohort of patients during a period of compliance with administration of deuterated arachidonic acid, an ester thereof, or a prodrug thereof, and (iii) measuring the difference between the natural rate of disease progression and the rate during the period of compliance, and dividing the difference by the natural rate of disease progression. The numerical value is then normalized by multiplying by 100.

In one embodiment, whether a therapeutic concentration of deuterated-arachidonic acid has been reached in neurons is measured using a reporter cell. In an embodiment, the reporter cells are red blood cells. In the case of red blood cells, a concentration of D-arachidonic acid of at least about 3% based on the total number of arachidonic acid, including deuterated arachidonic acid, contained in the red blood cells has been found to correlate with therapeutic results. Alternatively, the therapeutic concentration of arachidonic acid in the neurons can be extrapolated from the concentration of deuterated arachidonic acid in a reporter cell, such as red blood cells, as per the Examples below. See, e.g., U.S. Provisional Patent Application No. 63/177,794, filed Apr. 21, 2021, which is incorporated by reference in its entirety.

Preferably, the concentration of a specific deuterated arachidonic acid found in the neurons is sufficient to provide at least a 30% reduction in the rate of disease progression in a patient. As demonstrated in the Examples below, the concentration of deuterated arachidonic acid in red blood cells can be correlated to that found in the spinal fluid from which neurons obtain their cellular components. In turn, the neurons acquire arachidonic acid from the spinal fluid and, as such, there is a direct corollary between the concentration in the spinal fluid and that in the motor neurons. Therefore, the concentration of deuterated arachidonic acid in red blood cells acts as a proxy for the concentration in the motor neurons.

In one embodiment, the patients are placed on a diet that restricts intake of excessive amounts of linoleic acid, arachidonic acid, and/or other PUFA compounds in order to avoid insufficient uptake of the deuterated arachidonic acid by the body. Generally, dietary components that contribute to excessive amounts of PUFA consumed are restricted. Such dietary components include, for example, fish oil pills, products that contain high levels of PUFAs, such as salmon; patients on conventional feeding tubes may also have excessive PUFA intake. In a preferred embodiment, the methods described herein include both the dosing regimen described above as well as placing the patients on a restrictive diet that avoids excessive ingestion of PUFA components.

In one embodiment, there is provided a method for reducing the rate of disease progression in a patient suffering from a neurodegenerative disease treatable with 7,7,10,10,13,13-D6-arachidonic acid, which method comprises administering 7,7,10,10,13,13-D6-arachidonic acid or an ester thereof to the patient with a dosing regimen that comprises a primer or loading dosing and a maintenance dosing schedule which comprise:
a) said primer or loading dosing component comprises administering to said patient a dose of 7,7,10,10,13,13-D6-arachidonic acid or an ester thereof in a sufficient amount and for a period of time to allow for reduction in the rate of disease progression within no more than about 45 days from dosing;
b) subsequently following said primer or loading dose, initiating a maintenance dosing to said patient said dosing comprises an amount of 7,7,10,10,13,13-D6-arachidonic acid or ester thereof in an amount sufficient to maintain the concentration of 7,7,10,10,13,13-D6-arachidonic acid in the motor neurons wherein the amount of 7,7,10,10,13,13-D6-arachidonic acid or ester thereof administered in said maintenance dose is less than the amount administered in said primer or loading dose; and optionally:
c) monitoring the concentration of 7,7,10,10,13,13-D6-arachidonic acid in the patient to ensure that the patient is maintaining a therapeutic concentration of 7,7,10,10,13,13-D6-arachidonic acid; and
d) increasing the dosing of 7,7,10,10,13,13-D6-arachidonic acid or an ester thereof when said concentration of 7,7,10,10,13,13-D6-arachidonic acid is deemed to be less than a therapeutic amount.

### BRIEF DESCRIPTION OF THE DRAWINGS

FIG. 1 is a graph showing the percent of 13,13-D2-Arachidonic Acid in red blood cells (RBC) and spinal fluid (SF) at the indicated time points after start of treatment with 11,11-D2-Linoleic Acid.

### DETAILED DESCRIPTION

This disclosure is directed to methods for treating neurodegenerative diseases to significantly slow the rate of disease progression in a patient. In one embodiment, the methods described herein include a dosing regimen that is sufficient to provide a therapeutic level of deuterated arachidonic acid in the motor neurons. In another embodiment, the methods described herein comprise a daily or periodic primer dose that accelerates delivery of deuterated arachidonic acid to the diseased neurons of the patient. This primer dose is continued for a sufficient period of time to achieve a therapeutic concentration of a deuterated arachidonic acid in vivo. At that point, a daily or periodic maintenance dose is employed to maintain the therapeutic concentration of the deuterated arachidonic acid.

Prior to discussing this technology in more detail, the following terms will first be defined. Terms that are not defined are given their definition in context or are given their medically acceptable definition.

The terminology used herein is for the purpose of describing particular embodiments only and is not intended to be limiting of the invention. As used herein, the singular forms “a”, “an” and “the” are intended to include the plural forms as well, unless the context clearly indicates otherwise.

As used herein, the term “optional” or “optionally” means that the subsequently described event or circumstance can or cannot occur, and that the description includes instances where the event or circumstance occurs and instances where it does not.

As used herein, the term “about” when used before a numerical designation, e.g., temperature, time, amount, concentration, and such other, including a range, indicates approximations which may vary by (+) or (−) 15,% 10%, 5%, 1%, or any subrange or subvalue there between. Preferably, the term “about” when used with regard to a dose amount means that the dose may vary by +/−10%.

As used herein, the term “comprising” or “comprises” is intended to mean that the compositions and methods include the recited elements, but not excluding others.

As used herein, the term “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention.

As used herein, the term “consisting of” shall mean excluding more than trace elements of other ingredients and substantial method steps. Embodiments defined by each of these transition terms are within the scope of this invention.

As used herein, arachidonic acid has the numbering system as described below:

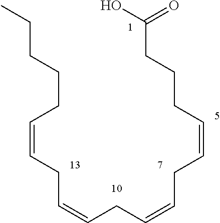 [SMILES: CCCCC/C=C\C/C=C\C/C=C\C/C=C\CCCC(=O)O]

where each of positions 7, 10 and 13 are bis-allylic positions within the structure.

As used herein and unless the context dictates otherwise, the term “deuterated arachidonic acid or an ester thereof” refers to any deuterated arachidonic acid, including 7,7,10,10,13,13-D6-arachidonic acid, or a C₁-C₆ alkyl ester, glycerol ester (including monoglycerides, diglycerides and triglycerides), sucrose esters, phosphate esters, and the like. The particular ester group employed is not critical provided that the ester group is pharmaceutically acceptable (non-toxic and biocompatible). The term “deuterated arachidonic acid” or “7,7,10,10,13,13-D6-arachidonic acid” includes a population (composition) of compounds that, on average, comprise very high levels of deuteration at positions 7, 10 and 13, as well as, on average, some or modest levels of deuteration at 4 and 16 positions. Such compositions can be prepared by catalytic processes described, for example, in U.S. Pat. No. 10,730,821, which is incorporated herein by reference in its entirety. For example, a composition of deuterated arachidonic acid may include greater than about 80% deuteration (on average) at bis-allylic sites, and modest (no more than about 40% on average) at mono-allylic sites. For example, the composition may include greater than about 80%, 85%, 90%, 95%, 96%, 97%, 98%, or 99% deuteration (on average) at bis-allylic sites of arachidonic acid. The composition may include less than about 40%, 35%, 30%, 20%, or 10% deuteration (on average) at mono-allylic sites of arachidonic acid. In one embodiment, the composition includes greater than about 95% deuteration (on average) at bis-allylic sites, and less than about 35% deuteration (on average) at mono-allylic sites.

As used herein and unless the context dictates otherwise, the term “an ester thereof” refers to a C₁-C₆ alkyl esters, glycerol esters (including monoglycerides, diglycerides and triglycerides), sucrose esters, phosphate esters, and the like. The particular ester group employed is not critical provided that the ester is pharmaceutically acceptable.

As used herein, the term “phospholipid” refers to any and all phospholipids that are components of the cell membrane. Included within this term are phosphatidylcholine, phosphatidylethanolamine, phosphatidylserine, and sphingomyelin. In the neurons, the cell membrane is enriched in phospholipids comprising arachidonic acid.

As used herein, the term “pathology of a disease” refers to the cause, development, structural/functional changes, and natural history associated with that disease. The term “natural history” means the progression of the disease in the absence of treatment per the methods described herein.

As used herein, the term “reduced rate of disease progression” means that the rate of disease progression is attenuated after initiation of treatment as compared to the patient's natural history. In one case, the rate of reduction in disease progression using the methods described herein results in a percentage reduction of at least 25% lower or at least 30% lower at a time point, e.g. 1 month to 24 months, e.g., 3 or 6 months, after initiation of therapy when compared to the natural history of the patient.

The term “therapeutic concentration” means a concentration of a deuterated arachidonic acid that reduces the rate of disease progression by at least 25% or at least 30%. Since obtaining the concentration of a deuterated arachidonic acid in the neurons or in the spinal fluid of a patient is either not feasible or optimal, the therapeutic concentration is based on the concentration of either deuterated arachidonic acid found in red blood cells as provided in the Examples below. Accordingly, any reference made herein to a therapeutic concentration of deuterated arachidonic acid is made by evaluating its concentration in red blood cells.

Alternatively, the reduction in the rate of disease progression is confirmed by a reduction in the downward slope (flattening the curve) of a patient's relative muscle functionality during therapy as compared to the downward slope found in the patient's natural history. Typically, the differential between the downward slope measured prior to treatment and the slope measured after at least 90 days from initiation of treatment has a flattening level of at least about 30%. So, a change of 7.5 degrees (e.g., a downward slope of 25 degrees during the natural history that is reduced to a downward slope of 17.5 degrees provides for a 40% decrease in the slope). In any case, the reduction in downward slope evidence that the patient has a reduced rate of disease progression due to the therapy.

As used herein, the term “patient” refers to a human patient or a cohort of human patients suffering from a neurodegenerative disease treatable by administration 7,7,10,10,13,13-D6-arachidonic acid or an ester thereof.

As used herein, the term “primer or loading amount” refers to an amount of a deuterated arachidonic acid or an ester thereof that is sufficient to provide for a reduced rate of disease progression within at least about 45 days after initiation of administration and preferably within 30 days. The amount so employed is loaded such that the patient has a stabilized rate of disease progression within this time period. When less than a loading amount is used, it is understood that such can provide therapeutic results but will not achieve the same level of reduction in disease progression. Given the progressive nature of neurodegenerative diseases, those dosing regimens that achieve the best reduction in the rate of disease progression are preferred as they are associated with the patient having less loss of muscle functionality over a given period of time.

The methods described herein are based on the discovery that the primer doses of deuterated arachidonic acid or an ester thereof employed to date are well tolerated by patients and provide for rapid onset to a therapeutic level in vivo to provide for a reduced and stabilized rate of disease progression.

As used herein, the term “maintenance dose” refers to a dose of deuterated arachidonic acid, e.g. 7,7,10,10,13,13-D6-arachidonic acid, or an ester thereof that is less than the primer dose and is sufficient to maintain a therapeutic concentration of deuterated arachidonic acid in the cell membrane of red blood cells and, hence, in the cell membrane of motor neurons, so as to retain a stable rate of disease progression.

As used herein, the term “periodic dosing” refers to a dosing schedule that substantially comports to the dosing described herein. Stated differently, periodic dosing includes a patient who is compliant at least 75 percent of the time over a 30-day period and preferably at least 80% compliant. In embodiments, the dosing schedule contains a designed pause in dosing. For example, a dosing schedule that provides dosing 6 days a week is one form of periodic dosing. Another example is allowing the patient to pause administration for from about 3 or 7 or more days, e.g. due to personal reasons, provided that the patient is otherwise at least 75 percent compliant.

The term “cohort” refers to a group of at least 2 patients whose results are to be averaged.

As used herein, the term “pharmaceutically acceptable salts” of compounds disclosed herein are within the scope of the methods described herein and include acid or base addition salts which retain the desired pharmacological activity and is not biologically undesirable (e.g., the salt is not unduly toxic, allergenic, or irritating, and is bioavailable). When the compound has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, trimethylamine, pyridine, picoline, ethanolamine, diethanolamine, triethanolamine) or basic amino acids (e.g., arginine, lysine, and ornithine). Such salts can be prepared in situ during isolation and purification of the compounds or by separately reacting the purified compound in its free base or free acid form with a suitable acid or base, respectively, and isolating the salt thus formed.

The phrase “excessive amounts of PUFAs,” “excessive PUFA intake,” and the like refer to intake of total PUFAs (e.g., total amount of PUFAs consumed per day) that result in reduced uptake of deuterated arachidonic acid compared to a diet lower in total PUFA intake. In embodiments, the patient is on a diet that restricts intake of PUFA compounds. The amount of PUFAs that can be consumed by a patient is variable, depending on numerous factors such as the patient's health, weight, age, other medications being taken, liver function, metabolism, and the like.

### Pathology

The discovery of several aldehydes that easily reacted with sulfhydryl groups, resulting in the inhibition of vital metabolic processes, led to the association of polyunsaturated fatty acid peroxidation as a component of the pathology of many of neurodegenerative diseases (Schauenstein, E.; Esterbauer, H. Formation and properties of reactive aldehydes. Ciba Found. Symp. (67):225-244; 1978). Whether as a primary cause of disease or a secondary consequence, such lipid peroxidation is attributed to oxidative stress, which leads to neural death and this implicated in the progression of a number of neurodegenerative diseases.

The oxidative stress responsible for such peroxidation is due to an imbalance between routine production and detoxification of reactive oxygen species (“ROS”) that leads to an oxidative attack on the lipid membrane of cells. The lipid membrane as well as the endoplasmic reticulum and mitochondria of motor neurons are highly enriched in arachidonic acid (a 20-carbon chain polyunsaturated fatty acid (“PUFA”) having 4 sites of cis-unsaturation). Separating each of these 4 sites are 3 bis-allylic methylene groups. These groups are particularly susceptible to oxidative damage due to ROS, and to enzymes such as cyclooxygenases, cytochromes and lipoxygenases, as compared to allylic methylene and methylene groups.

Moreover, once a bis-allylic methylene group in one arachidonic acid is oxidized by a ROS, a cascade of further oxidation of other arachidonic acid groups in the lipid membrane occurs. This is because a single ROS generates oxidation of a first arachidonic acid component through a free radical mechanism which, in turn, can oxidize a neighboring arachidonic acid through the same free radical mechanism which yet again can oxidize another neighboring arachidonic acid in a process referred to as lipid chain auto-oxidation. The resulting damage includes a significant number of oxidized arachidonic acid components in the cell membrane.

Oxidized arachidonic acids negatively affect the fluidity and permeability of cell membranes in motor neurons. In addition, they can lead to oxidation of membrane proteins as well as being converted into a large number of highly reactive carbonyl compounds. The latter include reactive species such as acrolein, malonic dialdehyde, glyoxal, methylglyoxal, etc. (Negre-Salvayre A, et al. Brit. J. Pharmacol. 2008; 153:6-20). But the most prominent products of arachidonic acid oxidation are alpha, beta-unsaturated aldehydes such as 4-hydroxynon-2-enal (4-HNE; formed from n-6 PUFAs like LA or AA), and corresponding ketoaldehydes (Esterfbauer H, et al. Free Rad. Biol. Med. 1991; 11:81-128. As noted above, these reactive carbonyls cross-link (bio)molecules through Michael addition or Schiff base formation pathways leading which continues the underlying pathology of the disease.

### Disease Progression

When a patient is diagnosed with a specific neurodegenerative disease, the clinician evaluates that patient's rate of disease progression by assessing the patient's loss of functionality in the absence of therapy as described herein. That rate is referred to as the “natural history” of the disease and is typically measured by standardized tests that measure the extent of a patient's functionality over a set period of time. For example, in the case of ALS, there is a standard test referred to as ALSFRS-R which determines the rate of loss of muscle functionality over time and this is used to measure disease progression. This test has 12 components each of which are measured on a 0 (worse) to 4 (best) scale. The ability of a drug to attenuate the rate of disease progression evidences its efficacy. Even a modest reduction in the rate of functionality loss is considered significant.

Heretofore, the treatment of a variety of neurodegenerative diseases employed deuterated 11,11-D2-linoleic acid or an ester thereof, including those in a lipid bilayer form, to stabilize polyunsaturated fatty acids against ROS. Examples of such treatments are found in: WO 2011/053870, WO 2012/148946, and WO 2020/102596, each of which is incorporated herein by reference in its entirety.

Each of these documents discloses the in vivo conversion of a portion of 11,11-D2-linoleic acid to 13,13-D2-arachidonic acid which is then incorporated into the motor neurons to stabilize these neurons from oxidative damage. The in vivo accumulation of 13,13-D2-arachidonic acid occurs over weeks if not months until a therapeutic concentration is achieved.

Still further, the dosing regimen employed must address the patient's need for rapid onset of therapy especially given that loss of functionality is typically very quick and quite often well before the end stage of the disease which typically ranges from about 2 to 5 years after diagnosis. Hence, any therapy for treating such neurodegenerative diseases must provide meaningful therapy within about 45 days and preferably within about a month or less after the start of therapy thereby retaining as much of the patient's functionality as possible and furthermore providing for substantial reductions in the rate of disease progression.

### Compound Preparation

Likewise, 7,7,10,10,13,13-D6-arachidonic acid are described, for example, in U.S. Pat. No. 10,730,821 which is incorporated herein by reference in its entirety. Other deuterated arachidonic acid compounds are known in the art.

Specifically, deuterated arachidonic acids can be prepared by catalytic processes as described in U.S. Pat. No. 10,577,304 which patent is incorporated herein by reference in its entirety. Those processes provide for substantially complete deuteration at the bis-allylic sites with some deuteration at non-bis-allylic sites and primarily at the mono-allylic sites. In general, deuteration at the 3 bis-allylic methylene sites converts the 6 hydrogen atoms to deuterium with greater than a 90% efficiency—meaning that in a population of deuterated arachidonic acid, the 6 bis-allylic hydrogen atoms have on average greater than 5.4 deuterium atoms. In addition, no more than about 35% of the total number of hydrogen atoms in arachidonic acid (excluding any ester portion) are replaced with deuterium. Since there are 32 hydrogen atoms in arachidonic acid, the percent of deuterium in these compounds ranges from about 15% to no greater than 35%. It is understood that when the defined term “deuterated arachidonic acid” recites D6 arachidonic acid, such includes arachidonic acid having greater than 5.4 deuterium atoms on average at the bis-allylic sites and a range of deuteration of from about 15% to no greater than 35% based on the number of hydrogen atoms present on non-deuterated arachidonic acid including the proton on the carboxylic acid.

One example, would be 90% deuteration at the bis-allylic sites and 15% deuteration at the mono-allylic sites with deuteration at the remaining sites being at the natural abundance. This provides for an average of 5.4 deuterium atoms at the bis-allylic sites and on average 0.6 deuterium atoms at the mono-allylic sites with a negligible amount of deuterium at the remaining sites to provide for a D6-composition.

One example would be 90% deuteration at the bis-allylic sites and 15% deuteration at the mono-allylic sites with deuteration at the remaining sites being at the natural abundance. This provides for an average of 5.4 deuterium atoms at the bis-allylic sites and on average 0.6 deuterium atoms at the mono-allylic sites with a negligible amount of deuterium at the remaining sites to provide for a composition defined as 7,7,10,10,13,13-D6-arachidonic acid.

Another example would be 80% deuteration at the bis-allylic sites and 10% deuteration at the mono-allylic sites with deuteration at the remaining sites being at the natural abundance. This provides for an average of 4.8 deuterium atoms at the bis-allylic sites and on average 0.4 deuterium atoms at the mono-allylic sites with a negligible amount of deuterium at the remaining sites to provide for a composition defined as 7,7,10,10,13,13-D6-arachidonic acid.

As defined above, both of such deuteration patterns are included within the definition of deuterated arachidonic acid.

Esters of these deuterated fatty acids are prepared by conventional techniques well known in the art. Such esters may be derived from Cl-05 alkyl alcohols.

### Methodology—7,7,10,10,13,13-D6-Arachidonic Acid or Ester Thereof

As noted previously, the deuterated arachidonic acid or ester thereof that is administered to the patient is 7,7,10,10,13,13-D6-arachidonic acid or an ester thereof as that term is defined herein. Because the three of the bis-allylic carbon atoms on these compounds have substantially all of the hydrogens replaced with deuterium, these compounds will provide superior reduction in the reducing or eliminating lipid auto-oxidation. As such, these compounds will provide a meaningful reduction in the rate of progression of ALS. In a preferred embodiment, the D-6 arachidonic acids or esters thereof are delivered in a tiered manner comprising a first and second dosing component. The first dosing component, the primer dose, follows the protocol set forth above with the exception that the primer uses between about 0.05 to about 5 grams or about 0.5 and about 5 grams of 7,7,10,10,13,13-D6-arachidonic acid or an ester thereof daily or periodically. The maintenance dose that is employed is generally between about 30% and about 70% of the primer dose.

The methods described herein may administer 7,7,10,10,13,13-D6-arachidonic acid or an ester thereof to a patient in order to accumulate a therapeutic concentration of 7,7,10,10,13,13-D6-arachidonic acid for use in the methods described herein.

In one embodiment, 7,7,10,10,13,13-D6-arachidonic acid or ester thereof is administered to the patient in sufficient amounts to generate a concentration of 7,7,10,10,13,13-D6-arachidonic acid in red blood cells of at least about 0.5%, preferably at least 1%, more preferably at least 2%, and most preferably at least 3%, based on the total amount of arachidonic acid, including deuterated arachidonic acid, found therein. At any of these concentrations, the attending clinician can correlate that concentration to a therapeutic concentration of 7,7,10,10,13,13-D6-arachidonic acid in the neurons. The percentage of 7,7,10,10,13,13-D6-arachidonic acid compared to total arachidonic acid in red blood cells may be between about 0.5% and about 60%. In an embodiment, the percentage of 7,7,10,10,13,13-D6-arachidonic acid compared to total arachidonic acid in red blood cells may be between about 0.5% and about 50%, between about 0.5% and about 40%, between about 0.5% and about 30%, between about 0.5% and about 20%, between about 0.5% and about 15%, between about 0.5% and about 10%, between about 0.5% and about 5%, between about 0.5% and about 4%, or between about 0.5% and about 3%. In an embodiment, the percentage of 7,7,10,10,13,13-D6-arachidonic acid compared to total arachidonic acid in red blood cells may be between about 1% and about 50%, between about 3% and about 40%, between about 5% and about 30%, between about 5% and about 20%, or between about 5% and about 15%.

In one embodiment, such administration comprises the use of a dosing regimen that includes two dosing components. The first dosing component comprises a primer dose of 7,7,10,10,13,13-D6-arachidonic acid or an ester thereof. The second dosing component comprises a maintenance dose of 7,7,10,10,13,13-D6-arachidonic acid or an ester thereof, wherein the amount of 7,7,10,10,13,13-D6-arachidonic acid or an ester thereof in said second dosing component is less than that in the first dosing component.

As to the primer dose, the amount of 7,7,10,10,13,13-D6-arachidonic acid or an ester thereof employed is designed to provide rapid onset of therapy. Such therapy is measured by a reduction in the disease progression of neurodegenerative diseases as described below. In an embodiment, the primer dose takes into account the various complicating factors, such as the amount of PUFAs consumed by the patient in a given day, as well as the general turnover rate of lipids (half-life) in the patient's neurons.

Regarding this last point, the lipid components of neurons are not static but, rather, are exchanged over time and have a finite half-life in the body. In general, only a fraction of the lipids components in the lipids are replaced each day. In the case of neurons, these cells are rich in arachidonic acid. The turnover of arachidonic acid in these membranes occurs from a stable pool of lipids comprising arachidonic acid in the spinal fluid. In turn, this stable pool is replaced and replenished over time by arachidonic acid included in the newly consumed lipids by the patient as part of the patient's diet as well as by biosynthesis of arachidonic acid from linoleic acid by the liver. In embodiments, the maintenance dose of the 7,7,10,10,13,13-D6-arachidonic acid is titrated such that the amount of 7,7,10,10,13,13-D6-arachidonic acid administered matches the rate of secretion from the body.

This disclosure is based on the discovery that given the above variables, the amount of 7,7,10,10,13,13-D6-arachidonic acid or ester thereof that is administered over time is selected so that the fatty acids contained in red blood cells comprise at least about 0.5% and preferably at least about 1%, and more preferably at least about 2%, and most preferably at least about 3% of 7,7,10,10,13,13-D6-arachidonic when tested at one (1) month after the start of therapy. At that level, the deuterated arachidonic acid concentration stabilizes the cell membrane and limits or prevents the cascade of lipid auto-oxidation. When so administered, there is a significant reduction in the progression rate of the neurodegenerative disease being treated.

The methods described herein are also based, in part, on the discovery that when the lipid membrane of neurons is stabilized against lipid peroxidation (LPO), there is a substantial reduction in the progression of the neurodegenerative disease. This is believed to be due to the replacement of hydrogen atoms with deuterium atoms in arachidonic acid, rendering the deuterated arachidonic acid significantly more stable to ROS than the hydrogen atoms. As above, this stability manifests itself in reducing the cascade of lipid auto-oxidation and, hence, limiting the rate of disease progression.

In one embodiment, the methods described herein address this challenge by employing a dosing regimen which delivers 7,7,10,10,13,13-D6-arachidonic acid in amounts sufficient to provide for a therapeutic amount of deuterated arachidonic acid in the neurons. When so incorporated, the deuterated arachidonic acid reduces the degree of LPO which, in turn, effectively limits progression of ALS provided it is administered in appropriate amounts.

### Combinations

The therapy provided herein can be combined with conventional treatment of used with neurodegenerative diseases provided that such therapy is operating on an orthogonal mechanism of action relative to inhibition of lipid auto-oxidation. Suitable drugs for use in combination include, but not limited to, antioxidants such as edaravone, idebenone, mitoquinone, mitoquinol, vitamin C, or vitamin E provided that none of these anti-oxidants that are directed to inhibiting lipid auto-oxidation, riluzole which preferentially blocks TTX-sensitive sodium channels, conventional pain relief mediations, and the like,

### Pharmaceutical Compositions

The specific dosing of 7,7,10,10,13,13-D6-arachidonic acid, or an ester thereof is accomplished by any number of the accepted modes of administration. As noted above, the actual amount of the drug used in a daily or periodic dose per the methods of this disclosure, i.e., the active ingredient, is described in detail above. The drug can be administered at least once a day, preferably once or twice or three times a day.

The compositions and methods described herein are not limited to any particular composition or pharmaceutical carrier, as such may vary. In general, compounds described herein will be administered as pharmaceutical compositions by any of a number of known routes of administration. However, orally delivery is preferred typically using tablets, pills, capsules, and the like. The particular form used for oral delivery is not critical but due to the large amount of drug to be administered, a daily or periodic unit dose is preferably divided into subunits having a number of tablets, pills, capsules, and the like. In one particularly preferred embodiment, each subunit of the daily or periodic unit dose contains about 1 gram of the drug. So, a daily or periodic unit dose of 9 grams of the drug is preferably provided as 9 sub-unit doses containing about 1 gram of the drug. Preferably, the unit dose is taken in one, two or three settings but, if patient compliance is enhanced by taking the daily or periodic unit dose over 2 or 3 settings per day, such is also acceptable.

Pharmaceutical dosage forms of a compound as disclosed herein may be manufactured by any of the methods well-known in the art, such as, by conventional mixing, tableting, encapsulating, and the like. The compositions as disclosed herein can include one or more physiologically acceptable inactive ingredients that facilitate processing of active molecules into preparations for pharmaceutical use.

The compositions can comprise the drug in combination with at least one pharmaceutically acceptable excipient. Acceptable excipients are non-toxic, aid administration, and do not adversely affect the therapeutic benefit of the claimed compounds. Such excipient may be any solid, liquid, or semi-solid that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Other suitable pharmaceutical excipients and their formulations are described in Remington's Pharmaceutical Sciences, edited by E. W. Martin (Mack Publishing Company, 18th ed., 1990).

The compositions as disclosed herein may, if desired, be presented in a pack or dispenser device each containing a daily or periodic unit dosage containing the drug in the required number of subunits. Such a pack or device may, for example, comprise metal or plastic foil, such as a blister pack, a vial, or any other type of containment. The pack or dispenser device may be accompanied by instructions for administration including, for example, instructions to take all of the subunits constituting the daily or periodic dose contained therein.

The amount of the drug in a formulation can vary depending on the number of subunits required for the daily or periodic dose of the drug. Typically, the formulation will contain, on a weight percent (wt %) basis, from about 10 to 99 weight percent of the drug based on the total formulation, with the balance being one or more suitable pharmaceutical excipients. Preferably, the compound is present at a level of about 50 to 99 weight percent.

In preferred embodiment, the drug is encapsulated inside a capsule without the need for any pharmaceutical excipients such as stabilizers, antioxidants, colorants, etc. This minimizes the number of capsules required per day by maximizing the volume of drug in each capsule.

### EXAMPLES

This invention is further understood by reference to the following examples, which are intended to be purely exemplary of this invention. This invention is not limited in scope by the exemplified embodiments, which are intended as illustrations of single aspects of this invention only. Any methods that are functionally equivalent are within the scope of this invention. Various modifications of this invention in addition to those described herein will become apparent to those skilled in the art from the foregoing description and accompanying FIGURES. Such modifications fall within the scope of the appended claims. In these examples, the following terms are used herein and have the following meanings. If not defined, the abbreviation has its conventional medical meaning.
D2-AA=13,13-D2-Arachidonic Acid
D6-AA=D6-arachidonic acid as defined herein
AA=Arachidonic Acid
ALSFRS-R=Revised ALS Functional Rating Scale
CNS=Central Nervous System
CSF=Cerebral Spinal Fluid
D2-LA=11,11-D2-Linoleic Acid (aka “drug”)
LA=Linoleic Acid
PK=Pharmacokinetics
RBC=Red Blood Cells
SAE=Serious Adverse Events

### Example 1—Determination of AA Concentrations in RBCs and Spinal Fluid/Neurons in a Single Patient

This example is intended to illustrate how to measure D6-AA in blood. The example utilizes D2-AA as proxy in order to establish the principle. Specifically, this example determines the relative concentration of D2-AA in the CSF and in RBCs in order to determine if there is a correlation between these two concentrations. Specifically, a patient was continuously provided with a daily dose of 9 grams of D2-LA ethyl ester over about a six-month period. Periodic samples of blood and SF were taken and the concentration of both D2-LA and D-2AA in both the RBCs and the SF were measured. In all cases, the D2-AA was obtained by deacylation of the ethyl ester of linoleic acid in the gastrointestinal tract followed by conversion of D2-LA in vivo to D2-AA.

TABLE 1

| | Concentration | Concentration | Ratio of D2-LA |
|---|---|---|---|
| | of D2-LA | of D2-AA | to D2-AA |
| Time | in SF | in SF | in SF |
| 1 month | 19.8% | 8% | 2.5:1 |

The results found in Table 1 show that the concentration of D2-AA in the cerebral spinal fluid is already 8% based on the amount of arachidonic acid+deuterated arachidonic acid.

Next, Table 2 shows that the concentration of D2-LA and D2-AA in the RBCs at 3 months and 6 months for the same patient.

TABLE 2

| | Concentration | Concentration | Ratio of D2-LA |
|---|---|---|---|
| | of D2-LA | of D2-AA | to D2-AA |
| Time | in RBCs | in RBCs | in RBCs |
| 3 months | 34.7% | 11.8% | 2.9:1 |
| 6 months | 34.5 | 16.7 | 2.1:1 |

Note here that the concentration of D2-AA in RBC's at 3 months is less than that at 6 months evidencing the incremental increase in D2-AA over time. Moreover, the ratio of D2-LA to D2-AA changes from 2.9:1 at 3 months to 2.1:1 at 6 months. In one embodiment, the ratio of D2-LA to D2-AA in RBCs at 3 and 6 months is represented as 2.5:1+/−0.4 which corresponds favorably to that found in Table 1.

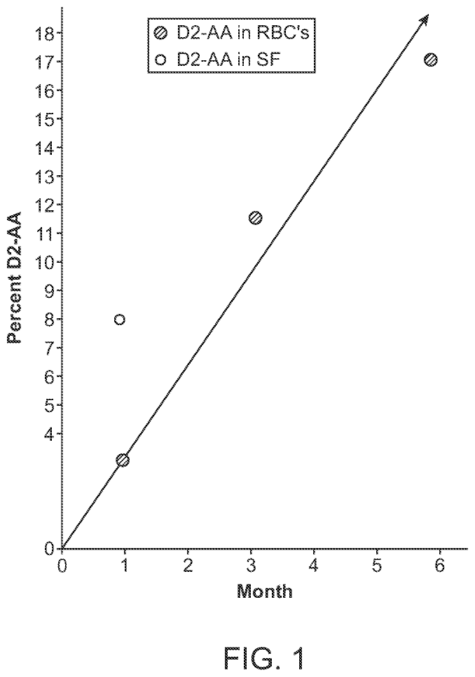

Since the amount of D2-AA is increasing over time in an incremental fashion based on the in vivo conversion of D2-LA, one can assume a fairly linear rate of increase. This is shown in FIG. 1, where the solid line is set by the concentrations of D2-AA at 3 months and 6 months and then extrapolated back to start of therapy (0 months). The value for the D2-AA in RBC's at 1 month is estimated from this relationship. The amount shown for 1 month in the CSF is also provided (open circle).

Based on the above, one can see that the data to date suggests that the amount D2-AA at 1 month in RBCs would be about 3 percent as compared to 8% for the amount of D2-AA in the SF. Accordingly, this data suggests that the concentration the body shunts more of the AA (including D2-AA) into the CSF (and hence the neurons) as compared to RBCs and likely other reporter cells.

This data establishes that the concentration of 13,13-D2-arachidonic acid in red blood cells can be extrapolated to that in the CSF, thereby providing a basis to correlate the concentration of 7,7,10,10,13,13-D6-arachidonic acid in RBCs to that in the CSF.

### Example 2—Control of LPS Induced Inflammation

It is well understood that inflammation plays a significant role in many neurodegenerative diseases. This example evaluates the impact of 13,13-D2-arachidonic acid relative to D6-arachidonic acid as it relates to inflammation as a corollary to LPO.

Lipopolysaccharide (LPS) administration is known to promote inflammation through various mechanisms including secretion of pro-inflammatory cytokines, eicosanoids and induction of ROS. This example employed LPS to ascertain the extent of inflammation arising from ROS induced oxidation of H-AA versus D-AA in the lungs of mice. Specifically, four groups of mice were used. The first group was control mice treated with H-LA (non-deuterated linoleic acid) control mice. The second group of mice received a 6-week course of D-LA. It is understood that in vivo conversion of a portion of both H-LA and D-LA occurs to provide for AA and 13,13-D2-AA respectively. The third group of mice received a 6-week course of H-AA (non-deuterated arachidonic acid). The fourth group of mice received a 6-week course of D6-AA.

All groups then received a single intranasal administration of LPS to induce acute lung inflammation. The degree of the inflammatory response was based on the interalveolar septa distance where the larger the distance of the septa, the greater the degree of inflammation. The animals were sacrificed and the interalveolar septa distance was measured. Table 1 provides an average degree of spatial distance for the interalveolar septa for the results of all groups.

TABLE 1

| | H-LA | D-LA | H-AA | D6-AA |
|---|---|---|---|---|
| Interalveolar | 14.2 um | 10.7 um | 9.1 um | 4.1 um |
| space | | | | |

The above results evidence about a 25% reduction in the spatial distance for the interalveolar septa for the mice treated with D-AA (by administration of the D2-LA prodrug) relative to those treated with H-LA. However, the mice treated D6-AA had almost a 60% reduction in the same spatial distance evidencing the benefits of D6-AA in treating inflammation. Moreover, this suggests that D6-AA provides at least greater than 2× the benefit than that of D2-AA.

### Comparative Example A

Patients suffering from ALS were treated with D2-LA over a period of time. The patients were given different dosing amounts of D2-LA and for different dosing periods but did not follow the dosing protocol described herein. Some patients were provided 2 grams of 11,11-D-2 LA per day as opposed to the loading dose of 9 grams per day.

Functional scores for each of the patients (ALSFRS-R results) at the end of therapy were compared to the natural history scores at the start of therapy. Based on this comparison, the rate of decline changed from an annualized rate of −14.2+/−4.4 per year pre-treatment to −7.6+/−1.4 during treatment or a 46% reduction (p=0.07, paired t-test for within-subject change in slope).

### Comparative Example B

This example illustrates the reduction in the rate of disease progression in patients with ALS treated by the dosing methods using D2-LA. Specifically, a cohort of 3 patients was placed on a dosing regimen consisting of a first dosing component (primer dose) of about 9 grams of D2-LA ethyl ester daily for a period of at least 30 days and then all three patients were transitioned to a second dosing component (maintenance dose) of 5 grams of D2-LA ethyl ester.

The functionality of each of the patients was evaluated periodically using the ALSFRS-R protocol. The patients continued on the dosing regimen for a period of 6 months (patient A) or 1 year (patient B) or for 9 months (patient C). Patient C died at the end of 9 months and his death was attributed to factors other than ALS cardiomyopathy. Before initiation of therapy, the natural history of each patient in the cohort was determined and an average annual rate of functional decline was measured at 21.

The annualized progression of the disease as measured by an average annual rate of functional decline for all three patients starting at the time that dosing began and terminating at the end of the dosing regimen and then annualized as described above was measured as 2.1. Using the formula described above, one obtains the following:

(21−2.1)/21×100=90% annualized average reduction in the rate of disease progression.

The specific values for each of the three members of the cohort are as follows in Table 5:

TABLE 5

| | | Functional |
|---|---|---|
| | NH Rate | Rate Decline |
| Patient | of Decline | During Therapy |
| A | −16 | −3 |
| B | −31 | −2 |
| C | −16 | −1.3 |

NH = Natural History

These results substantiate a very significant rate of reduction in the disease progression using the dosing regimen as described herein. These results also substantiate that transitioning patients from a primer dose to a maintenance dose maintains the beneficial stabilization in the rate of decline.

Patients treated with 9 gm of D2-LA per day for about 1 month followed by 5 gm of D2-LA per day thereafter evidence about a 90% reduction in the rate of disease progression, compared to the 46% rate of reduction in the single dose study (Comparative Example A).

This establishes that the dosing regimen described herein provides for a significant benefit to patients in their reduction in the rate of disease progression.

### CONCLUSION

The above data in Comparative Examples A and B establish that
D2-AA generated in vivo when properly dosed at 9 grams per day will significantly reduce the rate loss of functionality due to a neurodegenerative disease as compared to that when improperly dosed;
about 10% of D2-LA is converted in vivo to D2-AA as reported in the literature provided that the amount of PUFAs consumed is limited; and
Example 2 indicates that D6-AA is about 2+x more active than D2-AA.
   Based on the above, 9 grams of D2-LA provides for only 10% (or 0.9 gm per day) of D2-AA. D6-AA is at least 2 times more potent than D2-AA. As the amount of D2-LA is reported in U.S. Ser. No. 17/169,271 as ranging to about 7 grams per day, one can surmise that about 0.30 grams or more of D6-AA will provide the same benefit as that used for 7 grams of D2-LA.

1. A method for reducing neurodegenerative disease progression in a patient, the method comprising:
administering an effective amount of a deuterated arachidonic acid, an ester thereof, or a prodrug thereof, to reduce said disease progression in said patient;
wherein a therapeutic concentration of deuterated arachidonic acid in the motor neurons is sufficient to reduce disease progression, wherein said reduction is measured to be at least about 30% reduction relative to the rate of disease progression during the natural history of the patient.

2. The method of claim 1, wherein said disease is amyotrophic lateral sclerosis, Huntington's Disease, progressive supernuclear palsy (PSP), Friedreich's ataxia, APO-e4 Alzheimer's Disease, corticobasal disorder (CBD), frontotemporal dementia (FTD), nonfluent variant primary progressive aphasia (nfvPPA), other tauopathies, and late onset Tay-Sachs.

3. The method of claim 1, wherein the therapeutic concentration of deuterated arachidonic acid is determined by its concentration in red blood cells.

4. The method of claim 1, wherein a percent reduction in the rate of disease progression is determined by:
measuring a natural rate of disease progression in a patient or an average natural rate of disease progression in a cohort of patients;
measuring the rate of disease progression in said patient or cohort of patients during a period of compliance with the administering step; and
calculating the difference between the natural rate and the rate during the period of compliance, dividing the difference by the rate of disease progression during the natural history of the patient, and multiplying by 100.

5. The method of claim 1, wherein the deuterated arachidonic acid or ester thereof is 13,13-D2-arachidonic acid.

6. The method of claim 5, wherein the therapeutic concentration of 13,13-D2-arachidonic acid is determined by its concentration in red blood cells.

7. The method of claim 6, wherein the concentration of 13,13-D2-arachidonic acid in red blood cells is at least about 3% based on the total number of fatty acids contained in the red blood cells.

8. The method of claim 1, wherein the deuterated arachidonic acid or ester thereof is 7,7,10,10,13,13-D6-arachidonic acid or ester thereof.

9. The method of claim 8, wherein the therapeutic concentration of 7,7,10,10,13,13-D2-arachidonic acid is determined by its concentration in red blood cells.

10. The method of claim 9, wherein the concentration of 7,7,10,10,13,13-D6-arachidonic acid in the red blood cells is at least about 0.5% based on the total number of fatty acids contained therein.

11. A method for reducing disease progression of a neurodegenerative disease treatable with 7,7,10,10,13,13-D6-arachidonic acid or an ester thereof in an adult patient, the method comprising:
administering 7,7,10,10,13,13-D6-arachidonic acid or ester thereof to the patient with a dosing regimen that comprises a primer or loading dose and a maintenance dose thereby reducing said disease progression in said patient, wherein:
a) said primer or loading dose comprises periodic administration of from about 0.5 grams to about 5 grams of 7,7,10,10,13,13-D6-arachidonic acid or ester thereof per day, wherein said primer dose is continued for about 24 days to about 45 days; and
b) subsequent to the completion of the primer or loading dose, administering said maintenance dose of about 30% to about 70% of the primer or loading dose of 7,7,10,10,13,13-D6-arachidonic acid or ester thereof per day thereof such that the rate of disease progression is reduced,
wherein the neurodegenerative disease is mediated at least in part by lipid peroxidation of polyunsaturated fatty acids in neurons of the patient.

12. The method of claim 11, which further comprises:
c) monitoring the concentration of 7,7,10,10,13,13-D6 arachidonic acid in the patient to ensure that the patient maintains a therapeutic concentration of said 7,7,10,10,13,13-D6 arachidonic acid; and
d) increasing the dosing of 7,7,10,10,13,13-D6 arachidonic acid or an ester thereof when said concentrations of 7,7,10,10,13,13-D6-arachidonic acid are deemed to be less than a therapeutic concentration.

13. The method of claim 12, which further comprises restricting the patient's consumption of excessive dietary polyunsaturated fatty acids during said primer or loading dose and said maintenance dose.

14. The method of claim 11, wherein said neurodegenerative disease is amyotrophic lateral sclerosis, Huntington's Disease, progressive supernuclear palsy (PSP), Friedreich's ataxia, APO-e4 Alzheimer's Disease, corticobasal disorder (CBD), frontotemporal dementia (FTD), nonfluent variant primary progressive aphasia (nfvPPA), other tauopathies, and late onset Tay-Sachs.

15. The method of claim 11, wherein the therapeutic concentration of 7,7,10,10,13,13-D6 arachidonic acid is determined by its concentration in red blood cells.

16. The method of claim 15, wherein the concentration of 7,7,10,10,13,13-D6 arachidonic acid in red blood cells is at least about 0.5% based on the total amount of arachidonic acid, including deuterated arachidonic acid, present in the red blood cells.

17. The method according to claim 11, wherein 7,7,10,10,13,13-D6 arachidonic acid or an ester thereof is administered to the patient with a dosing regimen that comprises a primer or loading dose and a maintenance dose, the method further comprising:
a) administering said primer or loading dose to said patient at least about 0.5 grams of 11,11-D2-linoleic acid or an ester thereof wherein said first dosing component is continued for a period of from about 24 to about 45 days to provide a therapeutic concentration of 7,7,10,10,13,13-D6 arachidonic acid in red blood cells thereby reducing the rate of disease progression; and
b) subsequent to the period of from about 24 to about 45 days of administering said primer or loading dose administering the maintenance dose to said patient, said maintenance dose comprising about at least 0.15 grams of 7,7,10,10,13,13-D6 arachidonic acid or an ester thereof to maintain a therapeutic concentration of 7,7,10,10,13,13-D6 arachidonic acid provided that said maintenance dose is less than said primer dose.

18. The method of claim 17, which further comprises;
c) monitoring the concentration of 7,7,10,10,13,13-D6 arachidonic acid in the patient to ensure that the patient maintains a therapeutic concentration of 7,7,10,10,13,13-D6 arachidonic acid; and
d) increasing the dosing of 7,7,10,10,13,13-D6 arachidonic acid or an ester thereof when said concentrations of 7,7,10,10,13,13-D6 arachidonic acid is deemed to be less than a therapeutic concentration.

19. The method of claim 18, which further comprises restricting the patient's consumption of excessive dietary polyunsaturated fatty acids during said primer or loading dose and said maintenance dose.

20. A kit of parts comprising a set of capsules each comprising a partial loading dose of deuterated arachidonic acid or an ester thereof, such that two or more of said capsules comprise a complete loading dose per day.

21. A kit of parts comprising a set of capsules each comprising a partial loading dose of deuterated arachidonic acid or an ester thereof, such that nine of said capsules comprise a complete loading dose per day.

22. A kit of parts comprising a set of capsules each comprising a partial maintenance dose of deuterated arachidonic acid or an ester thereof, such that two or more of said capsules comprise a complete maintenance dose per day.

23. A kit of parts comprising a set of capsules each comprising a partial maintenance dose of deuterated arachidonic acid or an ester thereof, such that five of said capsules comprise a complete maintenance dose per day.
